(19) United States
(12) Patent Application Publication
(10) Pub. No.: US 2020/0038099 A1
(43) Pub. Date: Feb. 6, 2020

(54) TREATMENT SYSTEM

(71) Applicant: Olympus Corporation, Tokyo (JP)

(72) Inventor: Tsunetaka Akagane, Tokyo (JP)

(73) Assignee: Olympus Corporation, Tokyo (JP)

(21) Appl. No.: 16/600,414

(22) Filed: Oct. 11, 2019

Related U.S. Application Data
(63) Continuation of application No. PCT/JP2017/015297, filed on Apr. 14, 2017.

(51) Int. Cl. A61B 18/14

(52) U.S. Cl. CPC A61B 18/1445; A61B 2018/00083

(57) ABSTRACT

A treatment system includes an energy source apparatus having a high frequency power supply and a heater power supply. An elongated treatment tool is configured to be attached to the energy source apparatus to receive electrical energy. The elongated treatment tool includes a main body, a shaft, and a treatment portion all of which are attached to one another and are disposed on a longitudinal axis thereof. The treatment portion is used to grip a treatment target so as to apply appropriate gripping pressure to a point where the treatment target is to coagulate and to form a sealed region therein from an initial stage to a terminal stage of the treatment.

### CROSS-REFERENCE TO RELATED APPLICATION

This application is a continuation application of PCT Application No. PCT/JP 2017/015297 filed on Apr. 14, 2017, which is hereby incorporated by reference in its entirety.

### TECHNICAL FIELD

The disclosed technology relates to a generally to a treatment system, and more particularly, some embodiments relate to a treatment system for use with a treatment tool having electrodes and a heater.

### DESCRIPTION OF THE RELATED ART

US Patent Application Pub. No. 2016/0310207A1, for example, discloses a treatment tool for treating a biological tissue or biotissue by passing a high-frequency current through the biotissue and transferring heat from a heat generating body to electrodes. There is also disclosed a structure in the treatment tool for avoiding abutment between an electrode on one of a pair of treatment members and an electrode on the other treatment member.

For passing a high-frequency current through a treatment target in the biotissue to cause it to coagulate, it has been known that in order to obtain a suitable coagulating performance, it is necessary to continue applying an appropriate pressure to the position where the treatment target is to coagulate from initial to terminal stages of the treatment. For example, for passing an electric current through a blood vessel, for example, to form a sealed region therein, in order to obtain a suitable sealing performance, it is necessary to continue applying an appropriate pressure to the position where the blood vessel is to be sealed from initial to terminal stages of the treatment.

### BRIEF SUMMARY OF EMBODIMENTS

The disclosed technology has been made in view of the foregoing.

The disclosed technology is directed to an elongated treatment tool having a treatment portion disposed on a longitudinal axis thereof. The treatment portion includes a first treatment surface having a first electrically insulative surface and a first electrically conductive electrode extending along the longitudinal axis at a center of width of the first insulative surface. A second treatment surface having a second electrically insulative surface and a second electrically conductive electrode extending along the longitudinal axis of the second insulative surface. The second treatment surface is rotatable relatively with respect to the first treatment surface about a turn shaft perpendicular to the longitudinal axis and parallel to the widthwise directions perpendicular to the longitudinal axis. A heater is disposed on the first electrode for generating heat when supplied with electric power. When the second treatment surface is brought into abutment against the first treatment surface, the second electrically conductive electrode and the first electrically insulative surface abut against one another thereby to keep the first electrically conductive electrode and the second electrically conductive electrode spaced from one another.

Another aspect of the disclosed technology is directed to a treatment system having an energy source apparatus and an elongated treatment tool. The elongated treatment tool is configured to be attached to the energy source apparatus to receive electrical energy. The elongated treatment tool includes a treatment portion disposed on a longitudinal axis thereof and used to grip a treatment target such as a biological tissue. The treatment portion includes a first treatment surface having a first electrically insulative surface and a first electrically conductive electrode each of which extends along the longitudinal axis of the first electrically insulative surface. A second treatment surface having a second electrically insulative surface and a second electrically conductive electrode each of which extends along the longitudinal axis of the second insulative surface. The second treatment surface is rotatable with respect to the first treatment surface about a turn shaft perpendicular to the longitudinal axis. A heater is disposed on the first electrode to generate heat when supplied with electric power. When the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

A further aspect of the disclosed technology is directed to a treatment system includes an energy source apparatus having respective high frequency and heater power supplies and an elongated treatment tool configured to be attached to the energy source apparatus to receive electrical energy. The elongated treatment tool includes a main body, a shaft, and a treatment portion all of which are attached to one another and are disposed on a longitudinal axis thereof. The treatment portion is used to grip a treatment target so as to apply appropriate gripping pressure to a point where the treatment target is to coagulate and to form a sealed region therein from an initial stage to a terminal stage of the treatment. The treatment portion includes a first treatment surface having a first electrically insulative surface and a first electrically conductive electrode each of which extends along the longitudinal axis of the first electrically insulative surface. A second treatment surface having a second electrically insulative surface and a second electrically conductive electrode each of which extends along the longitudinal axis of the second insulative surface. The second treatment surface is rotatable with respect to the first treatment surface about a turn shaft perpendicular to the longitudinal axis. The heater is disposed on the first electrode to generate heat when supplied with the heater power supply. When the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view illustrating a bipolar treatment system according to first through third embodiments.

FIG. 2A is a schematic cross-sectional view, taken along line 2A-2A of FIG. 1, of a treatment portion of an elongated treatment tool according to the first embodiment in the system illustrated in FIG. 1.

FIG. 2B is a schematic view illustrating a state in which a first treatment surface of a first treatment member and a second treatment surface of a second treatment member of the treatment portion illustrated in FIG. 2A abut against each other.

FIG. 2C is an enlarged view of the treatment portion at a position indicated by the numeral reference 2C in FIG. 2B.

FIG. 3A is a schematic view illustrating the first treatment surface of the first treatment member in the treatment portion illustrated in FIG. 1.

FIG. 3B is a schematic view illustrating the second treatment surface of the second treatment member in the treatment portion illustrated in FIG. 1.

FIG. 3C is a schematic view illustrating a first modification of the first treatment surface of the first treatment member in the treatment portion illustrated in FIG. 1.

FIG. 3D is a schematic view illustrating a first modification of the second treatment surface of the second treatment member in the treatment portion illustrated in FIG. 1.

FIG. 3E is a schematic view illustrating a second modification of the first treatment surface of the first treatment member in the treatment portion illustrated in FIG. 1.

FIG. 3F is a schematic view illustrating a second modification of the second treatment surface of the second treatment member in the treatment portion illustrated in FIG. 1.

FIG. 4A is a schematic cross-sectional view, taken along line 2A-2A of FIG. 1, of a treatment portion of a treatment tool according to the second embodiment in the system illustrated in FIG. 1.

FIG. 4B is a schematic view illustrating a state in which a first treatment surface of a first treatment member and a second treatment surface of a second treatment member of the treatment portion illustrated in FIG. 4A abut against each other.

FIG. 5A is a schematic cross-sectional view, taken along line 2A-2A of FIG. 1, of a treatment portion of a treatment tool according to the third embodiment in the system illustrated in FIG. 1.

FIG. 5B is a schematic view illustrating a state in which a first treatment surface of a first treatment member and a second treatment surface of a second treatment member of the treatment portion illustrated in FIG. 5A abut against each other.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

It is an object of the disclosed technology to provide an elongated treatment tool that is capable of continuously applying an appropriate gripping pressure between treatment surfaces to a treatment target such as biological tissues from initial to terminal stages of the treatment.

Embodiments of the disclosed technology will be described hereinafter with reference to the drawings.

### First Embodiment

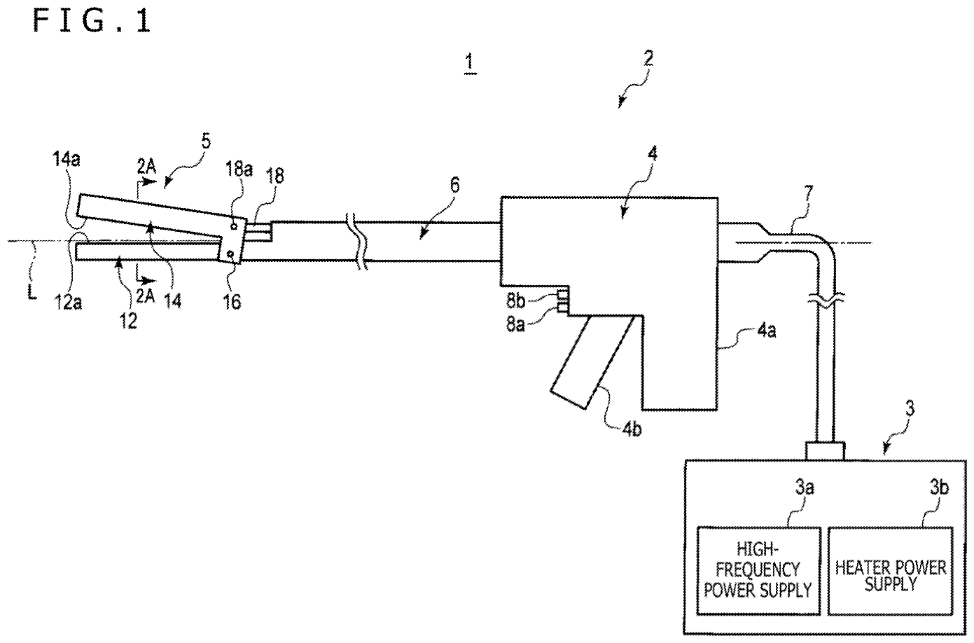

A first embodiment will be described hereinafter with reference to FIGS. 1 through 3B.

As illustrated in FIG. 1, a treatment system 1 has a treatment tool 2 and a power supply 3.

The elongated treatment tool 2 has a main body 4 and a treatment portion 5. A shaft 6 should preferably be disposed between the main body 4 and the treatment portion 5. The main body 4 is connected to a power supply 3 through a cable 7. The power supply 3 has a high-frequency power supply, i.e., an HF power supply, 3a and a heater power supply 3b for energizing a heater, i.e., a heat generating body, 25, to be described hereinafter, to generate heat. The power supply 3 is electrically connected to the treatment portion 5 through the main body 4.

The main body 4 has a fixed handle 4a integral with the main body 4 and a movable handle 4b movable toward and away from the fixed handle 4a.

A first switch 8a and a second switch 8b are disposed on the main body 4. According to the related art, when the first switch 8a on the main body 4 is pressed, the high-frequency power supply 3a supplies electric power to electrodes 24 and 34, coagulating a biotissue or sealing a blood vessel. Here, when the second switch 8b is pressed, for example, the high-frequency power supply 3a supplies electric power to the electrodes 24 and 34, and the heater power supply 3b supplies electric power to the heater 25 to generate heat, assisting in coagulating the biotissue or sealing the blood vessel with high-frequency output. The heater 25 is able to increase the temperature of an electrode surface 24a of the first electrode 24 with respect to the temperature thereof at the time an electric current is passed between the first electrode 24 and the second electrode 34, i.e., electrode members 42 and 44, thereby increasing the temperature of the biotissue or the blood vessel.

Generally, when electric power is supplied to the electrodes 24 and 34 to coagulate a biotissue or seal a blood vessel, the temperature of the biotissue or the blood vessel is held to a temperature up to approximately 100° C. When electric power is supplied to the heater 25 to incise a biotissue or a blood vessel, the temperature of the biotissue or the blood vessel can be increased to approximately several hundreds degrees Celsius. The temperature at which to incise a biotissue or a blood vessel is thus higher than the temperature at which to coagulate the biotissue or seal the blood vessel.

When the user releases the switch 8a, the power supply 3 stops supplying electric power to the first electrode 24 and the second electrode 34 of the treatment portion 5. Furthermore, when the user releases the switch 8b, the power supply 3 stops supplying electric power to the first electrode 24 and the second electrode 34 of the treatment portion 5 and also stops supplying electric power to the heater 25.

A structure in which the first switch 8a and the second switch 8b are disposed on the main body 4 and are operated by the user's finger will hereinafter be described by way of example. However, it is also preferable to employ a structure in which the switches are provided as foot switches connected to the power supply 3 and operable by the user's foot.

The treatment portion 5 has a first treatment member 12 and a second treatment member 14.

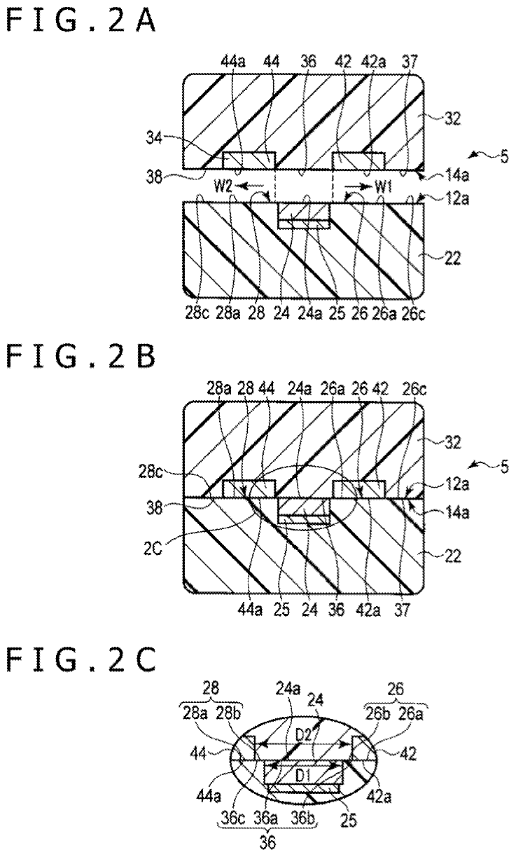

The main body 4 and the treatment portion 5 are disposed on an appropriate longitudinal axis L. The treatment portion 5 should preferably be longer in directions along the longitudinal axis L, i.e., longitudinal directions, than in widthwise directions W defined as directions perpendicular to the longitudinal axis L. In FIG. 2A, the widthwise directions W include a first direction indicated by the numeral reference W1 and a second direction indicated by the numeral reference W2. The first treatment member 12 and the second treatment member 14 are mutually angularly movably supported on a proximal end of the treatment portion 5 by a turn shaft 16. The turn shaft 16 should preferably extend perpendicularly to the longitudinal axis L and parallel to the widthwise directions W.

A drive shaft 18 is disposed between the main body 4 and the second treatment member 14 of the treatment portion 5. The drive shaft 18 is movable along the longitudinal axis L that represents a direction along which the treatment portion 5 extends from the main body 4. The drive shaft 18 is movable along the longitudinal axis L in ganged relation to the movable handle 4b as it moves. When the movable handle 4b is operated to move toward the fixed handle 4a of the main body 4, the drive shaft 18 is moved by a known mechanism to bring the second treatment member 14 that is coupled to a distal end 18a of the drive shaft 18 relatively toward the first treatment member 12. When the movable handle 4b is operated to move away from the fixed handle 4a, the drive shaft 18 is moved to bring the second treatment member 14 relatively away from the first treatment member 12.

The first treatment member 12 of the treatment portion 5 is attached to the main body 4. When the movable handle 4b of the main body 4 is operated, for example, the second treatment member 14 moves with respect to the first treatment member 12. Specifically, a first jaw 22 of the first treatment member 12 is movable toward and away from a second jaw 32 of the second treatment member 14. Alternatively, the treatment portion 5 may be of such a structure that when the main body 4 is operated, both the first treatment member 12 and the second treatment member 14 move relatively to the main body 4. The treatment portion 5 that is of the former structure will be described hereinafter. Whether the treatment portion 5 is of the former structure or the latter structure, the second jaw 32 is relatively movable toward and away from the first jaw 22.

As illustrated in FIGS. 1 through 3B, the first treatment member 12 of the treatment portion 5 has a first treatment surface, i.e., a gripper, 12a, and the second treatment member 14 has a second treatment surface, i.e., a gripper, 14a. The first treatment surface 12a of the first treatment member 12 faces the second treatment member 14. The second treatment surface 14a of the second treatment member 14 faces the first treatment member 12. The first treatment surface 12a and the second treatment surface 14a face each other. When the second treatment member 14 is angularly moved about the axis of the turn shaft 16 with respect to the first treatment member 12, the first treatment surface 12a and the second treatment surface 14a are moved toward and away from each other. The first treatment surface 12a and the second treatment surface 14a can grip a biotissue therebetween when they are moved toward each other. The first treatment surface 12a and the second treatment surface 14a can abut against each other when there is no biotissue present therebetween. Therefore, the treatment portion 5 of the treatment tool 2 according to the present embodiment can increase a gripping pressure on a thin treatment target such as a blood vessel or the like, compared with a treatment portion of a treatment tool that is of such a structure that when a first treatment surface and a second treatment surface are brought closely to each other, a spacer is disposed therebetween to keep the first treatment surface and the second treatment surface out of abutment against each other. The first treatment surface 12a and the second treatment surface 14a release the biotissue when they are separated from each other.

FIG. 2A illustrates a cross section taken along line 2A-2A of FIG. 1. Consequently, FIG. 2A illustrates a cross section of the treatment portion 5 perpendicular to the longitudinal axis L and substantially parallel to the widthwise directions W.

The first treatment member 12 has the first treatment surface 12a that moves toward or abuts against and moves away from the second treatment surface 14a. The first treatment member 12 has the first jaw 22 and the first electrode 24. The first treatment member 12 includes the heater, i.e., the heat generating body, 25 that generates heat when supplied with electric power. According to the present embodiment, the heater 25 is disposed on a reverse side of the first electrode 24. The heater 25 is attached to the first electrode 24 at a position opposite the electrode surface 24a in the vicinity of the center thereof in the widthwise directions W perpendicular to the longitudinal axis L. The heater 25 is covered with a material that is heat-resistant, electrically insulative, and has good thermal conductivity. Therefore, when the heater 25 is energized to generate heat, it can transfer the heat through the first electrode 24 to the first electrode surface 24a. The first treatment surface 12a should preferably be formed as a planar surface. The second treatment member 14 has the second jaw 32 and the second electrode 34. The second treatment member 14 has the second treatment surface 14a that moves toward or abuts against and moves away from the first treatment surface 12a. The second treatment surface 14a should preferably be formed as a planar surface.

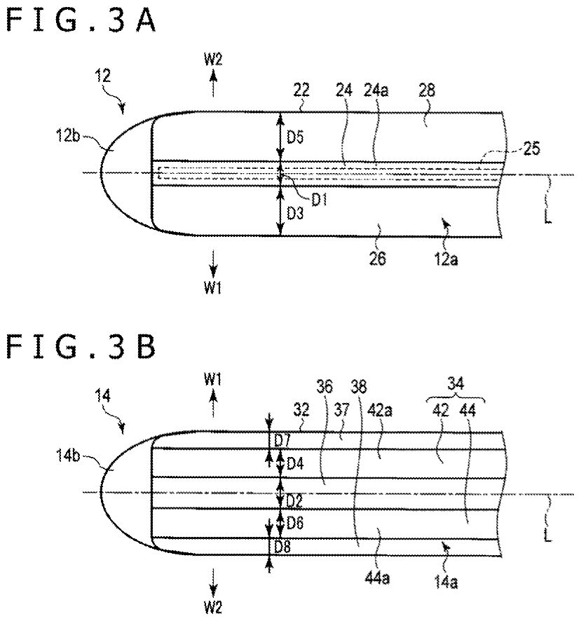

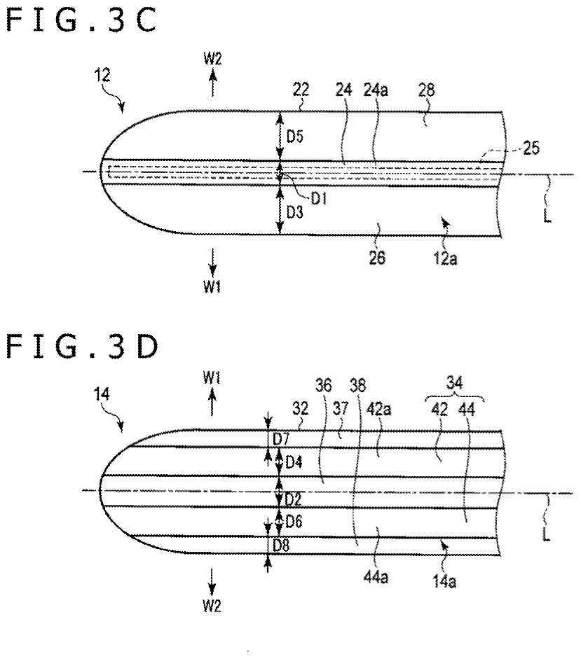

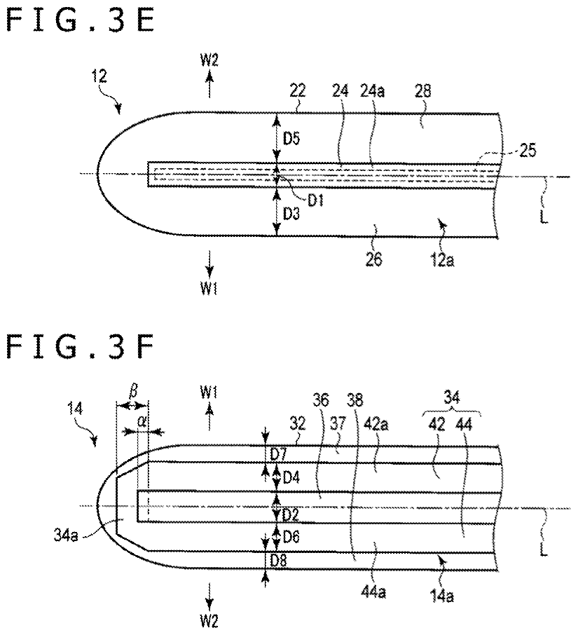

The first treatment surface 12a illustrated in FIG. 3A includes a distal-end surface 12b on a distal-end side thereof. The distal-end surface 12b should preferably be electrically insulative. The first treatment surface 12a and the distal-end surface 12b may lie or may not lie flush with each other. Similarly, the second treatment surface 14a illustrated in FIG. 3B includes a distal-end surface 14b on a distal-end side thereof. The distal-end surface 14b should preferably be electrically insulative. The second treatment surface 14a and the distal-end surface 14b may lie or may not lie flush with each other.

The first jaw 22 and the second jaw 32 extend along the longitudinal axis L. If the first jaw 22 and the second jaw 32 are made of a metal material that is electrically conductive, then the first jaw 22 and the second jaw 32 should preferably be covered with a material that is electrically insulative. The first jaw 22 and the second jaw 32 themselves may be made of a material that is electrically insulative which has appropriate rigidity. The first jaw 22 and the second jaw 32 should preferably have appropriate heat resistance. The first electrode 24 and the second electrode 34 are made of a material that is electrically conductive. The first electrode 24 and the second electrode 34 are used as different poles. Because of the electric insulation described hereinbefore, an unexpected electric current is prevented from flowing from the first electrode 24 to the first jaw 22. Similarly, an unintentional electric current is prevented from flowing from the second electrode 34 to the second jaw 32.

The first treatment surface 12a extends along the longitudinal axis L. The first treatment surface 12a has a first electrode surface, i.e., a surface for applying a gripping pressure, 24a defined by the first electrode 24, and planar portions, i.e., first insulative surfaces, 26 and 28 that are electrically insulative. The first planar portion 26 is disposed on the first direction W1 side of the first electrode surface 24a. The second planar portion 28 is disposed on the second direction W2 side of the first electrode surface 24a. According to the present embodiment, the first planar portion 26 and the second planar portion 28 that are integral with the first jaw 22 will be described by way of example. However, the first planar portion 26 and the second planar portion 28 may be separate from the first jaw 22.

The planar portions, i.e., surfaces for applying a gripping pressure, 26 and 28 are made of a material that, when heat caused by a high-frequency current is applied to a treatment target, e.g., a blood vessel or a biotissue, prevents the treatment target from sticking to the planar portions 26 and 28. The material of which the planar portions 26 and 28 are made should preferably be resistant to heat at approximately several hundred degrees, for example. The planar portions 26 and 28 of the first treatment surface 12a should preferably be made of fluororesin, for example, that is electrically insulative, as that material.

As illustrated in FIG. 3A, the first electrode 24 extends along the longitudinal axis L at the center of the first treatment surface 12a in the widthwise directions W. The planar portions 26 and 28 extend parallel to the longitudinal axis L at positions off the position along the longitudinal axis L at the center of the first treatment surface 12a in the widthwise directions W. Therefore, the first treatment surface 12a has the electrode 24 at the center thereof in the widthwise directions W and the planar portions 26 and 28 outside of the electrode 24 in the widthwise directions W.

The second treatment surface 14a extends along the longitudinal axis L. The second treatment surface 14a has planar portions, i.e., second insulative surfaces, 36, 37, and 38 that are electrically insulative, and electrode surfaces, i.e., surfaces for applying a gripping pressure, 42a and 44a defined by a plurality of electrode members 42 and 44 into which the second electrode 34 is divided.

The planar portions, i.e., surfaces for applying a gripping pressure, 36, 37, and 38 are made of a material that, when heat caused by a high-frequency current is applied to a treatment target, e.g., a blood vessel or a biotissue, prevents the treatment target from sticking to the planar portions 36, 37, and 38. The material of which the planar portions 36, 37, and 38 are made should preferably be resistant to heat at approximately several hundred degrees, for example. The planar portions 36, 37, and 38 of the second treatment surface 14a should preferably be made of fluororesin, for example, that is electrically insulative, as that material.

As illustrated in FIG. 3B, the planar portion, i.e., the second insulative surface, 36 extends along the longitudinal axis L at the center of the second treatment surface 14a in the widthwise directions W. The electrode surfaces 42a and 44a extend parallel to the longitudinal axis L at positions off the position along the longitudinal axis L at the center of the second treatment surface 14a in the widthwise directions W. Therefore, the second treatment surface 14a has the planar portion 36 at the center thereof in the widthwise directions W and the electrode surfaces 42a and 44a outside of the planar portion 36 in the widthwise directions W.

The first electrode member 42 is disposed on the first direction W1 side of the planar portion 36. The second electrode member 44 is disposed on the second direction W2 side of the planar portion 36. The electrode members 42 and 44 of the second electrode 34 are of the same pole and kept at the same electrical potential.

The planar portion 37 is disposed on the first direction W1 side of the first electrode member 42. The planar portion 38 is disposed on the second direction W2 side of the second electrode member 44. Therefore, the second treatment surface 14a has the planar portion 36 at the center thereof in the widthwise directions W, the electrode surfaces 42a and 44a of the electrode members 42 and 44 outside of the planar portion 36 in the widthwise directions W, and the planar portions 37 and 38 outside of the electrode members 42 and 44 in the widthwise directions W.

The electrode surface 24a of the first treatment surface 12a faces the planar portion 36 of the second treatment surface 14a. The planar portion 26 of the first treatment surface 12a faces the electrode surface 42a of the second treatment surface 14a. The planar portion 28 of the first treatment surface 12a faces the electrode surface 44a of the second treatment surface 14a.

As illustrated in FIG. 2C, the first planar portion 26 has a first abutment surface, i.e., an electrode abutment surface, 26a for abutting against the first electrode surface 42a, and a second abutment surface, i.e., an insulation abutment surface, 26b for abutting against the planar portion 36. The first abutment surface 26a and the second abutment surface 26b are contiguous to each other. The second planar portion 28 has a third abutment surface, i.e., an electrode abutment surface, 28a for abutting against the second electrode surface 44a, and a fourth abutment surface, i.e., an insulation abutment surface, 28b for abutting against the planar portion 36. The third abutment surface 28a and the second abutment surface 28b are contiguous to each other.

The planar portion 36 of the second treatment surface 14a has a first abutment surface, i.e., an electrode abutment surface, 36a for abutting against the electrode surface 24a, a second abutment surface, i.e., an insulation abutment surface, 36b that is contiguous to the first abutment surface 36a, for abutting against the first planar portion 26, and a third abutment surface, i.e., an insulation abutment surface, 36c that is contiguous to the second abutment surface 36a, for abutting against the second planar portion 28.

The boundary between the electrode surface 24a and the second abutment surface 26b of the planar portion 26 and the boundary between the electrode surface 24a and the fourth abutment surface 28b of the planar portion 28 should preferably lie flush with each other. The boundary between the electrode surface 42a and the second abutment surface 36b of the planar portion 36 and the boundary between the electrode surface 44a and the third abutment surface 36c of the planar portion 36 should preferably lie flush with each other.

Although not illustrated, spaces may be defined between the electrode surface 24a and the second abutment surface 26b of the planar portion 26 and between the electrode surface 24a and the fourth abutment surface 28b of the planar portion 28. In addition, spaces may be defined between the electrode surface 42a and the second abutment surface 36b of the planar portion 36 and between the electrode surface 44a and the third abutment surface 36c of the planar portion 36.

The first planar portion 26 has a third abutment surface, i.e., an insulation abutment surface, 26c in addition to the first abutment surface 26a and the second abutment surface 26b. The first abutment surface 26a, the second abutment surface 26b, and the third abutment surface 26c are contiguous to one another. The third abutment surface 26c abuts against the planar portion 37 in a planar fashion. Therefore, when the first treatment surface 12a and the second treatment surface 14a abut against each other, there is no gap between the third abutment surface 26c and the planar portion 37. Consequently, when the second treatment surface 14a abuts against the first treatment surface 12a, the first treatment surface 12a and the second treatment surface 14a have abutment surfaces 26c and 37 in areas on the first direction W1 side outside of the centers thereof along the widthwise directions W.

The second planar portion 28 has a third abutment surface, i.e., an insulation abutment surface, 28c in addition to the first abutment surface 28a and the second abutment surface 28b. The first abutment surface 28a, the second abutment surface 28b, and the third abutment surface 28c are contiguous to one another. The third abutment surface 28c abuts against the planar portion 38 in a planar fashion. Therefore, when the first treatment surface 12a and the second treatment surface 14a abut against each other, there is no clearance between the third abutment surface 28c and the planar portion 38. Consequently, when the second treatment surface 14a abuts against the first treatment surface 12a, the first treatment surface 12a and the second treatment surface 14a have abutment surfaces 28c and 38 in areas on the second direction W2 side outside of the centers thereof along the widthwise directions W.

According to the present embodiment, for the sake of brevity, it is assumed that the first treatment surface 12a and the second treatment surface 14a have the same width in the widthwise directions W. With the first treatment surface 12a and the second treatment surface 14a in abutment against each other, a widthwise dimension D1 of the electrode surface 24a of the first treatment surface 12a is smaller than a widthwise dimension D2 of the planar portion 36 of the second treatment surface 14a. With the first treatment surface 12a and the second treatment surface 14a in abutment against each other, a widthwise dimension D3 of the planar portion 26 of the first treatment surface 12a is larger than a widthwise dimension D4 of the electrode surface 42a of the second treatment surface 14a. Similarly, with the first treatment surface 12a and the second treatment surface 14a in abutment against each other, a widthwise dimension D5 of the planar portion 28 of the first treatment surface 12a is larger than a widthwise dimension D6 of the electrode surface 44a of the second treatment surface 14a. The sum of a width D7 of the planar portion 37 and a width D4 of the electrode member 42 of the second electrode 34 is smaller than a width D3 of the planar portion 26. The sum of a width D8 of the planar portion 38 and a width D6 of the electrode member 44 of the second electrode 34 is smaller than a width D5 of the planar portion 28. Therefore, the length of the planar portions 26 and 28 of the first treatment surface 12a along the widthwise directions W is larger than the length of the second electrode 34 along the widthwise directions W. Moreover, the length of the planar portion 36 of the second treatment surface 14a along the widthwise directions W is larger than the length of the first electrode 24 along the widthwise directions W.

Next, operation of the treatment tool 2 according to the present embodiment will be described hereinafter.

The user of the treatment tool 2 moves the movable handle 4b of the main body 4 toward the fixed handle 4a until the second treatment surface 14a abuts against the first treatment surface 12a.

The first abutment surface 26a of the first planar portion 26 of the first treatment surface 12a abuts against the electrode surface 42a of the electrode member 42 of the second treatment surface 14a in a planar fashion. At this time, the first abutment surface 26a of the first planar portion 26 of the first treatment surface 12a abuts against the electrode surface 42a of the electrode member 42 of the second treatment surface 14a in either of the directions along the longitudinal axis L and the widthwise directions W perpendicular to the longitudinal axis L.

The third abutment surface 28a of the second planar portion 28 of the first treatment surface 12a abuts against the electrode surface 44a of the electrode member 44 of the second treatment surface 14a in a planar fashion. At this time, the third abutment surface 28a of the second planar portion 28 of the first treatment surface 12a abuts against the electrode surface 44a of the electrode member 44 of the second treatment surface 14a in either of the directions along the longitudinal axis L and the widthwise directions W perpendicular to the longitudinal axis L.

Therefore, the planar portions, i.e., first areas, 26 and 28 have the respective abutment surfaces 26a and 28a abutting respectively against the electrode members 42 and 44 of the second electrode 34 in a planar fashion.

The first abutment surface 36a of the planar portion, i.e., second area, 36 of the second treatment surface 14a abuts against the electrode surface 24a of the first treatment surface 12a in a planar fashion. At this time, the first abutment surface 36a of the planar portion 36 of the second treatment surface 14a abuts against the electrode surface 24a of the first treatment surface 12a in either of the directions along the longitudinal axis L and the widthwise directions W perpendicular to the longitudinal axis L.

Of the planar portion 26 of the first treatment surface 12a, the second abutment surface 26b that is closer to the center in the widthwise directions W abuts against the second abutment surface 36b, positioned toward the first direction W1 of the widthwise directions W, of the planar portion 36 of the second treatment surface 14a. Of the planar portion 28 of the first treatment surface 12a, the fourth abutment surface 28b that is closer to the center in the widthwise directions W abuts against the third abutment surface 36c, positioned toward the second direction W2 of the widthwise directions W, of the planar portion 36 of the second treatment surface 14a. In view of wobbling movements, etc. of the second treatment member 14 with respect to the first treatment member 12, the width, i.e., abutting area, between the second abutment surface 26b and the second abutment surface 36b and the width, i.e., abutting area, between the fourth abutment surface 28b and the third abutment surface 36c are set to appropriate values.

Consequently, the first treatment surface 12a has the planar portions, i.e., surfaces for applying a gripping pressure, 26 and 28 that include the abutment surfaces 26a and 28a for abutting against the second electrode 34, i.e., the electrode surfaces 42a and 44a in a planar fashion. Furthermore, the second treatment surface 14a has the planar portion, i.e., a surface for applying a gripping pressure, 36 for abutting against the planar portions 26 and 28, the planar portion 36 including the abutment surface 36a for abutting against the first electrode 24, i.e., the electrode surface 24a in a planar fashion.

Therefore, even when the first treatment surface 12a and the second treatment surface 14a are held in abutment against each other, the first electrode 24 and the second electrode 34 are disposed in positions spaced from each other. Specifically, the first electrode 24 and the second electrode 34 are spaced from each other in at least either the directions along the longitudinal axis L or the widthwise directions W perpendicular to the longitudinal axis L. Consequently, even when the first switch 8a is pressed to pass a high-frequency current between the first electrode 24 and the second electrode 34, a short circuit is prevented from developing between the first electrode 24 and the second electrode 34.

When the first treatment surface 12a and the second treatment surface 14a of the treatment portion 5 of the treatment tool 2 according to the present embodiment are held in abutment against each other, no gap is present in opening and closing directions, perpendicular to the longitudinal axis L and the widthwise directions W, of the first treatment surface 12a and the second treatment surface 14a. Therefore, even if a tissue gripped between the first treatment surface 12a and the second treatment surface 14a is a thin tissue, a gripping pressure is transmitted to the tissue.

Moreover, no spacer is present between the first treatment surface 12a and the second treatment surface 14a. Consequently, a gripping pressure acting on a biotissue as a treatment target between the first treatment surface 12a and the second treatment surface 14a is restrained from changing largely along the widthwise directions W. In addition, a biotissue as a treatment target is easily gripped in a larger area between the first treatment surface 12a and the second treatment surface 14a.

A treatment, i.e., an electrifying treatment, for passing a high-frequency current through a blood vessel, not illustrated, to form a sealed region therein, using the treatment portion 5 of the treatment tool 2 according to the present embodiment will be described by way of example hereinafter.

A blood vessel as a treatment target is gripped between the first treatment surface 12a and the second treatment surface 14a. The blood vessel is gripped while in contact with both the first treatment surface 12a and the second treatment surface 14a. At this time, the blood vessel extends out of the treatment portion 5 along the widthwise directions W, for example.

The blood vessel is gripped between the electrode surface 24a and the planar portion 36, between the abutment surface 26a and the electrode surface 42a, and between the abutment surface 28a and the electrode surface 44a. Therefore, the blood vessel is held in contact with both the electrode 24 of the first treatment surface 12a and the electrode 34 of the second treatment surface 14a, i.e., the electrode members 42 and 44, while kept under a gripping pressure. Respective paths through the blood vessel between the first electrode 24 and the electrode member 42 of the second electrode 34 and between the first electrode 24 and the electrode member 44 of the second electrode 34 are made short.

When the user presses the first switch 8a, electric power is supplied from the power supply 3 through the main body 4 of the treatment tool 2 to the first electrode 24 and the second electrode 34, applying a voltage between the first electrode 24 and the second electrode 34. A high-frequency current thus flows through the blood vessel gripped between the first electrode 24 and the second electrode 34. In other words, the high-frequency current is applied to a portion of the blood vessel as the treatment target where a sealed region is to be formed. At this time, heat caused by the high-frequency current is applied to not only positions near the electrode surfaces 42a and 44a of the electrode members 42 and 44, but also the blood vessel between the electrode surfaces 42a and 44a of the electrode members 42 and 44, between the electrode surface 24a and the electrode surfaces 42a and 44a of the electrode members 42 and 44. Therefore, the length of the blood vessel along a width D1 in the widthwise directions W of at least the electrode surface 24a can be affected by the heat caused by the high-frequency current. The blood vessel between the first electrode 24 and the second electrode 34, i.e., the electrode members 42 and 44 thereof, is progressively dehydrated and dried, and hence made thin by the electrifying treatment. At this time, the distance between the first treatment surface 12a and the second treatment surface 14a, i.e., the distance in the opening and closing directions, is reduced as the blood vessel becomes thinner.

It is known that obtaining a good sealing performance using the treatment tool 2 for performing an electrifying treatment on a blood vessel to form a sealed region therein depends upon not only the state of the blood vessel, but also the gripping pressure applied to the blood vessel.

The sealing performance for blood vessels is required to withstand an appropriate blood pressure of several hundreds mmHg, for example. Since the sealing performance is possibly subject to variations, it is preferable to set the sealing performance of the treatment tool 2 such that it can withstand a high blood pressure of 1000 mmHg, for example.

The first treatment surface 12a and the second treatment surface 14a of the treatment portion 5 of the treatment tool 2 according to the present embodiment are configured between themselves into a state able to abut against each other. Therefore, as the treatment to seal a blood vessel progresses and the blood vessel becomes progressively thinner, the gripping pressure on the blood vessel rises. When the treatment, i.e., the electrifying treatment, to seal the blood vessel is about to be finished, a maximum gripping pressure is applied to the blood vessel. Consequently, appropriate gripping pressures are continuously applied to the blood vessel from the initial to terminal stages of the treatment. Therefore, the blood vessel is well sealed using the spacerless and gapless treatment tool 2 in which the first treatment surface 12a and the second treatment surface 14a abut against each other. In other words, an appropriate sealed region is formed in the blood vessel.

The planar portion 37 is disposed outside of the electrode surface 42a of the electrode member 42 in the first direction W1 of the widthwise directions W. The planar portion 38 is disposed outside of the electrode surface 44a of the electrode member 44 in the second direction W2 of the widthwise directions W. The third abutment surface 26c abuts against the planar portion 37 in a planar fashion. The third abutment surface 28c abuts against the planar portion 38 in a planar fashion. Therefore, an appropriate gripping pressure is applied to a blood vessel gripped between the abutment surface 26c and the planar portion 37 and a blood vessel gripped between the abutment surface 28c and the planar portion 38. No energy is applied from the electrode members 42 and 44 to the blood vessel gripped between the abutment surface 26c and the planar portion 37 and the blood vessel gripped between the abutment surface 28c and the planar portion 38. Consequently, no heat is generated directly in the blood vessel gripped between the abutment surface 26c and the planar portion 37 and between the abutment surface 28c and the planar portion 38. Therefore, the heat caused by the treatment carried out by the treatment portion 5 is prevented from escaping out of the treatment portion 5 via the blood vessel gripped between the abutment surface 26c and the planar portion 37 and between the abutment surface 28c and the planar portion 38. The gripping pressure is applied to the blood vessel in the vicinity of widthwise outer edges of the treatment surfaces 12a and 14a. As the gripping pressure acting on the blood vessel between the abutment surface 26c and the planar portion 37 and between the abutment surface 28c and the planar portion 38 reduces the path of the heat, the heat is prevented from escaping outside in the widthwise directions W, i.e., out of the treatment portion 5. Therefore, the heat generated when the high-frequency current is passed through the blood vessel is prevented as much as possible from escaping out via the blood vessel and from thermally invading a biotissue outside of the treatment portion 5.

When heat is applied to a blood vessel to form a sealed region therein, the blood vessel may shrink toward the center thereof in the widthwise directions W. As the blood vessel shrinks, a force is applied to open the treatment surfaces 12a and 14a relatively to each other. Even in this case, a gripping pressure remains applied to the blood vessel between the abutment surface 26c and the planar portion 37 and between the abutment surface 28c and the planar portion 38 in the vicinity of the outer edges of the treatment surfaces 12a and 14a in the widthwise directions W. The gripping pressure between the first treatment surface 12a and the second treatment surface 14a can be increased as the electrifying treatment of the treatment target is in progress. Therefore, the blood vessel is prevented as much as possible from shrinking toward the center in the widthwise directions W. Therefore, the gripping pressure is kept applied to the blood vessel between the first treatment surface 12a and the second treatment surface 14a from the initial to terminal stages of the treatment. The gripping pressure between the first treatment surface 12a and the second treatment surface 14a prevents the biotissue as the treatment target from shrinking, i.e., from gathering toward the center in the widthwise directions W, as the treatment is in progress.

The example in which the treatment is performed by supplying electric power from the high-frequency power supply 3a to the electrodes 24 and 34 to form a sealed region in a blood vessel has been described hereinbefore. A similar treatment is carried out to coagulate a treatment target of a biotissue.

When the second switch 8b is pressed to treat a blood vessel, the high-frequency power supply 3a supplies electric power to the electrodes 24 and 34 and the heater power supply 3b supplies electric power to the heater 25. In case the treatment target is a blood vessel, a sealed region is formed in the blood vessel and the heater produced by the heater 25 is transferred to the electrode surface 24a of the electrode 24. Therefore, the heater 25 increases the temperature of the electrode surface 24a of the first electrode 24 with respect to the temperature thereof at the time an electric current is passed between the first electrode 24 and the second electrode 34, i.e., the electrode members 42 and 44. Even though the blood vessel has been made thin, an appropriate gripping pressure has been applied between the first treatment surface 12a and the second treatment surface 14a. The heat from the heater 25 is applied from the electrode surface 24a to the blood vessel, assisting in sealing the blood vessel with the high-frequency output. By setting the temperature generated by the heater 25 to an appropriate value, for example, the region of the blood vessel that has been sealed by the high-frequency output can be incised by the heat transferred from the electrode surface 24a.

When the heat from the heater 25 is transferred via the electrode surface 24a of the electrode 24, the blood vessel may shrink toward the center thereof in the widthwise directions W. Even in this case, a gripping pressure remains applied to the blood vessel between the abutment surface 26c and the planar portion 37 and between the abutment surface 28c and the planar portion 38 in the vicinity of the outer edges of the treatment surfaces 12a and 14a in the widthwise directions W. Therefore, the blood vessel is prevented as much as possible from shrinking toward the center in the widthwise directions W. Therefore, the gripping pressure is kept applied to the blood vessel between the first treatment surface 12a and the second treatment surface 14a from the initial to terminal stages of the treatment.

As described hereinbefore, the treatment tool 2 according to the present embodiment deserves to be commented as follows:

If there is no biotissue present between the first treatment surface 12a and the second treatment surface 14a, then there is no gap between the first treatment surface 12a and the second treatment surface 14a. Therefore, when a biotissue is gripped between the first treatment surface 12a and the second treatment surface 14a, the treatment surfaces 12a and 14a apply a gripping pressure to the treatment target at all times regardless of whether the biotissue is thin or is made thin by an electrifying treatment. Therefore, an electric current can be passed between the first electrode 24 and the second electrode 34 while the biotissue is being strongly compressed therebetween.

At this time, since there is no gap present between the first treatment surface 12a and the second treatment surface 14a, the first treatment surface 12a and the second treatment surface 14a can grip the biotissue that is thin or is made thin by an electrifying treatment, in a wider area thereof. Consequently, forces are less likely to concentrate on one location of the biotissue, preventing the biotissue from being incised unexpectedly during the treatment.

For forming a sealed region in a blood vessel, for example, the first treatment surface 12a and the second treatment surface 14a grip the blood vessel in a wider area thereof. Even if the blood vessel is thin or the blood vessel becomes progressively thinner as the treatment progresses, an appropriate gripping pressure can be applied to the blood vessel continuously from the initial to terminal stages of the electrifying treatment. Therefore, the sealed state of the sealed region of the blood vessel is stabilized. Moreover, the blood pressure resistance of the blood vessel, i.e., the difficulty with which the blood flows through the blood vessel, is increased by the sealed region.

Therefore, the treatment tool 2 according to the present embodiment is capable of continuously applying an appropriate gripping pressure between the treatment surfaces 12a and 14a to a treatment target such as a blood vessel, a biotissue, or the like that becomes thinner as an electrifying treatment progresses. Accordingly, the treatment portion 5 of the treatment tool 2 according to the present embodiment is able to increase the gripping pressure on a thin treatment target such as a blood vessel or the like, compared with a treatment portion of a treatment tool having such a structure that a spacer is disposed between a first treatment surface and a second treatment surface when they come close to each other, preventing the first treatment surface and the second treatment surface from abutting against each other.

In the vicinity of the outer edge of the treatment portion 5 of the treatment tool 2 according to the present embodiment, positioned away in the first direction W1 from the center in the widthwise directions W, the surfaces 26c and 37 that are insulative abut against each other in a planar fashion. In the vicinity of the outer edge of the treatment portion 5 of the treatment tool 2 according to the present embodiment, positioned away in the second direction W2 from the center in the widthwise directions W, the surfaces 28c and 38 that are insulative abut against each other in a planar fashion. Therefore, even when electric power is supplied from the power supply 3 to the treatment portion 5, no heat is directly produced in a blood vessel or a biotissue between the abutment surface 26c and the planar portion 37 and between the abutment surface 28c and the planar portion 38. Therefore, the heat generated by the treatment performed by the treatment portion 5 is prevented from escaping out of the treatment portion 5 via the blood vessel between the abutment surface 26c and the planar portion 37 and between the abutment surface 28c and the planar portion 38. In addition, a biotissue outside of the treatment portion 5 is prevented as much as possible from being invaded.

Between the surfaces 26c and 37 and between the surfaces 28c and 38, the gripping pressure increases as the treatment progresses. Consequently, even when the biotissue between the first treatment surface 12a and the second treatment surface 14a tends to shrink along the widthwise directions W, the biotissue is prevented as much as possible from gathering toward the center in the widthwise directions W by the gripping pressure between the surfaces 26c and 37 and between the surfaces 28c and 38. In other words, the biotissue is prevented as much as possible from gathering toward the center in the widthwise directions W in the treatment by the gripping pressure between the first treatment surface 12a and the second treatment surface 14a.

According to the present embodiment, the example in which the first treatment surface 12a has the single electrode surface 24a and the two planar portions, i.e., insulative surfaces, 26 and 28 and the second treatment surface 14a has the two electrode surfaces 42a and 44a and the single planar portion, i.e., insulative surface, 36 has been described hereinbefore. However, the first treatment surface 12a may have two electrode surfaces and a single insulative surface and the second treatment surface 14a may have a single electrode surface and two insulative surfaces. Therefore, the first treatment surface 12a and the second treatment surface 14a may have a single electrode member or a plurality of electrode members.

In the example illustrated in FIG. 3A, the distal-end surface 12b that is electrically insulative is disposed on the distal-end side of the first treatment surface 12a. Therefore, the distal end of the electrode surface 24a is positioned closer to the proximal end of the first treatment member 12 than the distal end thereof. In the example illustrated in FIG. 3B, the distal-end surface 14b is disposed on the distal-end side of the second treatment surface 14a. Therefore, the distal end of the planar portion 36 that faces the electrode surface 24a is positioned closer to the proximal end of the second treatment member 14 than the distal end thereof.

FIG. 3C illustrates a first modification of the first treatment surface 12a of the first treatment member 12. FIG. 3D illustrates a first modification of the second treatment surface 14a of the second treatment member 14.

As illustrated in FIG. 3C, the distal-end side of the first treatment surface 12a is free of the distal-end surface 12b (see FIG. 3A) that is electrically insulative. The distal end of the electrode surface 24a is aligned with the distal end of the first treatment member 12. In case the treatment surface 12a of the treatment member 12 is in the state illustrated in FIG. 3C, the distal-end side of the second treatment surface 14a is free of the distal-end surface 14b (see FIG. 3B) that is electrically insulative. The planar portion 36 that faces the electrode surface 24a is in an area including the distal end of the second treatment member 14 so as to abut against the electrode surface 24a illustrated in FIG. 3C. In this case, the electrode surfaces 42a and 44a have distal ends disposed in the area including the distal end of the second treatment member 14.

FIG. 3E illustrates a second modification of the first treatment surface 12a of the first treatment member 12. FIG. 3F illustrates a second modification of the second treatment surface 14a of the second treatment member 14.

As illustrated in FIG. 3E, the distal-end side of the first treatment surface 12a is free of the distal-end surface 12b (see FIG. 3A) that is electrically insulative. The distal end of the electrode surface 24a is positioned closer to the proximal end of the first treatment member 12 than the distal end thereof. In case the treatment surface 12a of the first treatment member 12 is in the state illustrated in FIG. 3E, the distal-end portion of the planar portion 36 of the second treatment surface 14a protrudes a distance a (>0) from the distal end of the electrode surface 24a of the first treatment surface 12a as illustrated in FIG. 3F. The electrode 34 that includes the electrode surfaces 42a and 44a has an electrode surface 34a that is contiguous in an area between the distal end of the planar portion 36 and the distal-end surface 14b that is electrically insulative. Therefore, the electrode surface 34a of the electrode 34 is substantially U-shaped on the second treatment surface 14a. A broken line near the distal end of the planar portion 36 illustrated in FIG. 3F represents a position that becomes closest to the distal end of the electrode surface 24a of the first treatment surface 12a when the first treatment surface 12a and the second treatment surface 14a are relatively closed. Therefore, when the first treatment surface 12a and the second treatment surface 14a are relatively closed, the distal end of the electrode surface 24a abuts against or is close to the planar portion 36 that is electrically insulative. The distal-end surface 14b that is electrically insulative is disposed on the distal-end side of the distal end of the electrode surface 34a, i.e., the electrode surfaces 42a and 44a. The distal end of the electrode surface 34a, i.e., the electrode surfaces 42a and 44a protrudes a distance β (>α>0) from the broken line near the distal end of the planar portion 36 illustrated in FIG. 3F. Therefore, the distal end of the second treatment member 14 is electrically insulative.

The treatment performance can be varied by the structure in the vicinity of the distal-end portion of the first treatment surface 12a side of the first treatment member 12 and in the vicinity of the distal-end portion of the second treatment surface 14a side of the second treatment member 14.

According to the first modification illustrated in FIGS. 3C and 3D, the treatment portion 5 is capable of incising a biotissue with substantially the entire lengths of the first treatment surface 12a and the second treatment surface 14a along the longitudinal axis L. For example, when the first treatment surface 12a and the second treatment surface 14a grip a biotissue in the vicinity of their distal ends along the longitudinal axis L, they can cut the biotissue progressively by small lengths. Therefore, the first treatment surface 12a and the second treatment surface 14a of the treatment portion 5 according to the first modification are useful in incising thin membranes, etc. that require detailed work.

According to the second modification illustrated in FIGS. 3E and 3F, even when the first treatment surface 12a and the second treatment surface 14a of the treatment portion 5 grip a biotissue in the vicinity of their distal ends, they cannot cut the biotissue. On the other hand, the first treatment surface 12a and the second treatment surface 14a can firmly grip a biotissue, for example, with suitable portions thereof between the distal and proximal ends thereof along the longitudinal axis L and roughly cut the biotissue. Furthermore, the portions of the first treatment surface 12a and the second treatment surface 14a of the treatment portion 5 in the vicinity of their distal ends function as regions for sealing a biotissue. Consequently, when the treatment portion 5 according to the second modification grips approximately one-half of a blood vessel, it can incise the blood vessel while preventing it from bleeding.

As described hereinbefore, the portion of the first treatment surface 12a of the first treatment member 12 in the vicinity of its distal-end portion and the portion of the second treatment surface 14a of the second treatment member 14 in the vicinity of its distal-end portion are not limited to the structures illustrated in FIGS. 3A and 3B. The portion of the first treatment surface 12a in the vicinity of its distal-end portion and the portion of the second treatment surface 14a in the vicinity of its distal-end portion may be, for example, of the structures illustrated in FIGS. 3C and 3D according to the first modification or the structures illustrated in FIGS. 3E and 3F according to the second modification. The portion of the first treatment surface 12a in the vicinity of its distal end portion and the portion of the second treatment surface 14a in the vicinity of its distal-end portion may be of other various shapes.

In the first embodiment described hereinbefore, the first treatment surface 12a and the second treatment surface 14a are illustrated as flat. However, the first treatment surface 12a and the second treatment surface 14a may not be flat, but may be curved.

### Second Embodiment

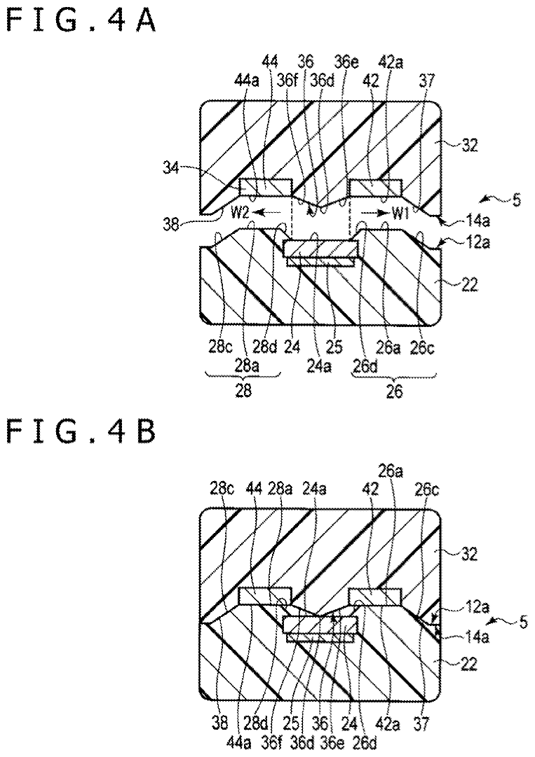

A second embodiment will be described hereinafter with reference to FIGS. 4A and 4B. The second embodiment is a modification of the first embodiment. Those parts of the second embodiment that are identical or have identical functions to those parts described in the first embodiment are denoted if at all possible by identical numeral references, and will not be described in detail hereinafter. This also holds true for a third embodiment to be described hereinafter. The structures according to the first through third embodiments can appropriately be combined with each other.

According to the first embodiment, the example in which the abutment surface 36a of the flat planar portion 36 of the second treatment surface 14a abuts against the electrode surface 24a of the first treatment surface 12a in a planar fashion has been described. According to the present embodiment, an example in which a planar portion 36 has a non-flat protrusion 36d and slanted surfaces 36e and 36f will be described.

According to the present embodiment, as illustrated in FIGS. 4A and 4B, each of the first treatment surface 12a and the second treatment surface 14a has recesses and projections.

The planar portion, i.e., the first insulative surface, 26 and the planar portion, i.e., the first insulative surface, 28 of the first treatment surface 12a protrude toward the second treatment surface 14a with respect to the electrode surface 24a of the electrode 24 that is disposed adjacent thereto on the central side in the widthwise directions W.

Specifically, the abutment surface, i.e., the electrode abutment surface, 26a of the planar portion 26 protrudes toward the second treatment surface 14a with respect to the electrode surface 24a of the electrode 24. The planar portion 26 has a slanted surface 26d lying between the abutment surface 26a and the electrode surface 24a and contiguous to the abutment surface 26a. The slanted surface 26d makes the abutment surface 26a of the planar portion 26 protrude toward the second treatment surface 14a with respect to the electrode surface 24a. Similarly, the abutment surface, i.e., the electrode abutment surface, 28a of the planar portion 28 protrudes toward the second treatment surface 14a with respect to the electrode surface 24a of the electrode 24. The planar portion 28 has a slanted surface 28d lying between the abutment surface 28a and the electrode surface 24a and contiguous to the abutment surface 28a. The slanted surface 28d makes the abutment surface 28a of the planar portion 28 protrude toward the second treatment surface 14a with respect to the electrode surface 24a. According to the present embodiment, therefore, the first treatment surface 12a is shaped as a non-flat surface.

The planar portion, i.e., the second insulative surface, 36 of the second treatment surface 14a protrudes toward the first treatment surface 12a with respect to the electrode surface 42a that is adjacent to the planar portion 36 in the first direction W1 of the widthwise directions W and the electrode surface 44a that is adjacent to the planar portion 36 in the second direction W2 of the widthwise directions W.

The planar portion 36 protrudes toward the electrode surface 24a of the first treatment surface 12a progressively from the outer sides toward the center in the widthwise directions W. According to the present embodiment, therefore, the second treatment surface 14a is shaped as a non-flat surface. Of the planar portion 36, a protrusive portion or crest indicated by the numeral reference 36d which protrudes most toward the first treatment surface 12a should preferably be positioned at the center in the widthwise directions W. Of the planar portion 36, the region between the protrusion 36d and the electrode surface 42a of the electrode member 42 is shaped as a slanted surface 36e. The region between the protrusion 36d and the electrode surface 44a of the electrode member 44 is shaped as a slanted surface 36f. The slanted surfaces 36e and 36f make the protrusion 36d of the planar portion 36 protrude toward the electrode surface 24a of the first treatment surface 12a. Therefore, the planar portion 36 has a substantially V-shaped cross section. The protrusion 36d should preferably extend continuously from nearly the distal end to nearly the proximal end of the second treatment surface 14a along the longitudinal axis L. The protrusion 36d can abut against the electrode surface 24a of the first treatment surface 12a.

When the protrusion 36d is held in abutment against the electrode surface 24a of the first treatment surface 12a, the abutment surface 26a of the planar portion 26 and the electrode surface 42a of the electrode member 42 abut against each other and the abutment surface 28a of the planar portion 28 and the electrode surface 44a of the electrode member 44 abut against each other.

The abutment surface 26c near the edge of the first treatment surface 12a in the first direction W1 of the widthwise directions W has at least a portion slanted with respect to the first direction W1. The abutment surface 28c near the edge of the first treatment surface 12a in the second direction W2 of the widthwise directions W has at least a portion slanted with respect to the second direction W2. The planar portion 37 near the edge of the second treatment surface 14a in the first direction W1 of the widthwise directions W has at least a portion slanted with respect to the first direction W1. The planar portion 38 near the edge of the second treatment surface 14a in the second direction W2 of the widthwise directions W has at least a portion slanted with respect to the second direction W2.

The abutment surface 26c of the first treatment surface 12a and the planar portion 37 of the second treatment surface 14a abut against each other in a planar fashion. The abutment surface 28c of the first treatment surface 12a and the planar portion 38 of the second treatment surface 14a abut against each other in a planar fashion.

As illustrated in FIGS. 4A and 4B, according to the present embodiment, the electrode surface 24a and the electrode surface 42a, and the electrode surface 24a and the electrode surface 44a do not face each other along the directions in which the first treatment surface 12a and the second treatment surface 14a are opened and closed, i.e., the directions perpendicular to both the longitudinal axis L and the widthwise directions W. The electrode surface 24a and the electrode surface 42a, and the electrode surface 24a and the electrode surface 44a may face each other along the directions in which the first treatment surface 12a and the second treatment surface 14a are opened and closed.

Next, operation of the treatment tool 2 according to the present embodiment will be described hereinafter.

According to the present embodiment, in the same way as described in the first embodiment, when the first switch 8a is pressed, the high-frequency power supply 3a supplies electric power to electrodes 24 and 34, coagulating a biotissue or sealing a blood vessel. When the second switch 8b is pressed, for example, the high-frequency power supply 3a supplies electric power to the electrodes 24 and 34, and the heater power supply 3b supplies electric power to the heater 25. Therefore, according to the present embodiment, an example in which when the second switch 8b is pressed, the heater power supply 3b supplies electric power to the heater 25 to cause the heater 25 to generate heat, incising a coagulated area immediately after the coagulated area is formed in a biotissue or incising a sealed area immediately after the sealed area is formed in a blood vessel will be described hereinafter.

When the second treatment surface 14a is brought into abutment against the first treatment surface 12a, the electrode surface 24a and the protrusion 36d abut against each other, the abutment surface 26a and the electrode surface 42a abut against each other in a planar fashion, the abutment surface 28a and the electrode surface 44a abut against each other in a planar fashion, the abutment surface 26c and the planar portion 37 abut against each other in a planar fashion, and the abutment surface 28c and the planar portion 38 abut against each other in a planar fashion. Furthermore, when the second treatment surface 14a is brought into abutment against the first treatment surface 12a, gaps are defined between the slanted surface 26d and the slanted surface 36e as well as the electrode surface 42a and between the slanted surface 28d and the slanted surface 36f as well as the electrode surface 44a.

Therefore, when the first switch 8a or the second switch 8b is pressed to cause a high-frequency current to flow between the first electrode 24 and the second electrode 34, a short circuit is prevented from developing between the first electrode 24 and the second electrode 34. Of the electrode surface 42a, the area closer to the center in the widthwise directions W faces the slanted surface 26d along the directions in which the first treatment surface 12a and the second treatment surface 14a are opened and closed. Of the electrode surface 44a, the area closer to the center in the widthwise directions W faces the slanted surface 28d along the directions in which the first treatment surface 12a and the second treatment surface 14a are opened and closed. The electrode surface 24a and the electrode surface 42a are close to each other, and the electrode surface 24a and the electrode surface 44a are close to each other.

When the second treatment surface 14a is brought into abutment against the first treatment surface 12a, the electrode surface 24a at the center in the widthwise directions W and the protrusion 36d abut against each other, the abutment surface 26a spaced from the center in the first direction W1 and the electrode surface 42a abut against each other, and the abutment surface 28a spaced from the center in the second direction W2 and the electrode surface 44a abut against each other. In particular, the abutment surface 26a and the electrode surface 42a, and the abutment surface 28a and the electrode surface 44a abut against each other in a planar fashion. Therefore, since the abutment surface 26a and the electrode surface 42a, and between the abutment surface 28a and the electrode surface 44a, of the first treatment surface 12a and the second treatment surface 14a of the treatment portion 5 of the treatment tool 2 according to the present embodiment, abut against each other in a planar fashion, there are no gaps therebetween along the directions in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

Furthermore, the abutment surface 26c and the planar portion 37 abut against each other in a planar fashion, and the abutment surface 28c and the planar portion 38 abut against each other in a planar fashion. Therefore, since the abutment surfaces 26a and 26c and the electrode surface 42a as well as the planar portion 37, and the abutment surfaces 28a and 28c and the electrode surface 44a as well as the planar portion 38 abut against each other in a planar fashion, there are no gaps therebetween along the directions in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

There are no spacers present between the abutment surfaces 26a and 26c and the electrode surface 42a as well as the planar portion 37, and between the abutment surfaces 28a and 28c and the electrode surface 44a as well as the planar portion 38. Therefore, the gripping pressure under which a biotissue as a treatment target is gripped along the widthwise directions W is restrained from varying greatly between the abutment surfaces 26a and 26c and the electrode surface 42a as well as the planar portion 37, and between the abutment surfaces 28a and 28c and the electrode surface 44a as well as the planar portion 38. Moreover, a biotissue as a treatment target can easily be gripped in a larger area between the abutment surfaces 26a and 26c and the electrode surface 42a as well as the planar portion 37, and between the abutment surfaces 28a and 28c and the electrode surface 44a as well as the planar portion 38.

While the first treatment surface 12a and the second treatment surface 14a of the treatment portion 5 of the treatment tool 2 according to the present embodiment are held in abutment against each other, therefore, there are regions in which no gaps are present along the directions in which the first treatment surface 12a and the second treatment surface 14a are opened and closed and which are perpendicular to the longitudinal axis L and the widthwise directions W. Consequently, even if a tissue gripped between the first treatment surface 12a and the second treatment surface 14a is a thin tissue, the gripping pressure is reliably transmitted to the tissue.

A treatment, i.e., an electrifying treatment, for passing a high-frequency current through a blood vessel, not illustrated, to form a sealed region therein, using the treatment portion 5 of the treatment tool 2 according to the present embodiment will be described by way of example hereinafter.

In the same manner as described in the first embodiment, a blood vessel as a treatment target is gripped between the first treatment surface 12a and the second treatment surface 14a. The blood vessel is gripped while in contact with both the first treatment surface 12a and the second treatment surface 14a.

There are gaps defined between the slanted surface 26d and the slanted surface 36e as well as the electrode surface 42a, and between the slanted surface 28d and the slanted surface 36f as well as the electrode surface 44a. A blood vessel is gripped between the electrode surface 24a and the protrusion 36d, between the abutment surface 26a and the electrode surface 42a, and between the abutment surface 28a and the electrode surface 44a. Therefore, the blood vessel is held in contact with both the electrode 24 of the first treatment surface 12a and the electrode 34 of the second treatment surface 14a while under the gripping pressure.

When the user presses the first switch 8a, electric power is supplied from the power supply 3 through the main body 4 of the treatment tool 2 to the first electrode 24 and the second electrode 34. Paths through the blood vessel between the first electrode 24 and the electrode member 42 of the second electrode 34 and between the first electrode 24 and the electrode member 44 of the second electrode 34 are short. Therefore, a high-frequency current flows through the blood vessel gripped between the first electrode 24 and the second electrode 34. Specifically, a high-frequency current is applied to a portion of the blood vessel as the treatment target where a sealed region is to be formed. At this time, heat caused by the high-frequency current is applied to not only positions near the electrode surfaces 42a and 44a of the electrode members 42 and 44, but also the blood vessel between the electrode surfaces 42a and 44a of the electrode members 42 and 44, between the electrode surface 24a and the electrode surfaces 42a and 44a of the electrode members 42 and 44. Therefore, a length of the blood vessel which is commensurate with the width D1 in the widthwise directions W of at least the electrode surface 24a is subjected to the heat caused by the high-frequency current. The blood vessel between the first electrode 24 and the second electrode 34 is progressively dehydrated and dried, and becomes thinner. At this time, the electrode surface 24a and the protrusion 36d become closer to each other, the abutment surface 26a and the electrode surface 42a become closer to each other in a planar fashion, and the abutment surface 28a and the electrode surface 44a become closer to each other in a planar fashion. Therefore, the distance between the first treatment surface 12a and the second treatment surface 14a becomes smaller as the blood vessel is thinner.

Consequently, the treatment portion 5 of the treatment tool 2 according to the present embodiment applies a maximum gripping pressure when it is about to finish the treatment to seal the blood vessel. Consequently, appropriate gripping pressures are continuously applied to the blood vessel from the initial to terminal stages of the treatment. Therefore, the blood vessel is well sealed using the spacerless and gapless treatment tool 2 in which the first treatment surface 12a and the second treatment surface 14a abut against each other in a planar fashion. In other words, an appropriate sealed region is formed in the blood vessel.

Appropriate gripping pressures are also continuously applied between the abutment surface 26c and the planar portion 37 and between the abutment surface 28c and the planar portion 38 from the initial to terminal stages of the treatment. In the treatment portion 5 of the treatment tool 2 according to the present embodiment, particularly, the area of the abutment surface 26c along the widthwise directions W and the area of the planar portion 37 along the widthwise directions W are made of not a simple flat surface, but a combination of surfaces. Similarly, the area of the abutment surface 28c along the widthwise directions W and the area of the planar portion 38 along the widthwise directions W are made of not a simple flat surface, but a combination of surfaces. Therefore, paths along which the heat generated when the high-frequency current is passed escapes outwardly through the blood vessel are made complex, making it difficult for the heat to escape outwardly, thus preventing the heat from invading a biotissue outside of the treatment portion 5 as much as possible.

The example in which the treatment is performed by pressing the first switch 8a to supply electric power from the high-frequency power supply 3a to the electrodes 24 and 34 to form a sealed region in a blood vessel has been described hereinbefore. A similar treatment is carried out to coagulate a treatment target of a biotissue.

Next, an example in which a sealed region is formed in a blood vessel and the formed sealed region is incised using the treatment tool 2 according to the present embodiment will be described hereinafter.

For forming a sealed region by performing an electrifying treatment on a blood vessel and incising the sealed region formed by the electrifying treatment, it has been known that a good incising performance using the treatment tool 2 depends on the temperature applied to the blood vessel in addition to the state of the blood vessel and the gripping pressure on the blood vessel. For incising the blood vessel, it is preferable to energize the heater 25 to generate heat and apply the heat at a temperature in excess of 100° C., e.g., approximately 200° C., through the electrode surface 24a to the blood vessel while an appropriate gripping pressure is being applied thereto.

The example illustrated in FIGS. 4A and 4B has been described on the assumption that the area of contact between the electrode surface 24a of the electrode 24 of the first treatment surface 12a and the protrusion 36d of the planar portion 36 of the second treatment surface 14a is appropriately small in the widthwise directions W. In this case, the sharper the shape of the protrusion 36d, the larger the pressure that the planar portion 36 of the second treatment surface 14a is able to apply to the biotissue per unit area. Therefore, the sharper the shape of the protrusion 36d, the easier it is for the planar portion 36 of the second treatment surface 14a to incise the biotissue. On the other hand, if a blood vessel is incised before a sealed region is formed therein, then since the blood vessel is likely to breed, the protrusion 36d is set to a suitable shape such as a blunt shape.

With the treatment portion 5 of the treatment tool 2 according to the present embodiment, the protrusion 36d applies a pressure to press the sealed region of the blood vessel against the electrode surface 24a. Even when the blood vessel is progressively thinner at the center in the widthwise directions W, the treatment portion 5 continues to apply an appropriate gripping pressure between the electrode surface 24a and the protrusion 36d. In this state, the heat generated by the heater 25 is transferred to the electrode surface 24a of the electrode 24. Therefore, while the appropriate pressure is being applied to the sealed region of the blood vessel, the temperature of the sealed region is increased to a temperature in excess of 100° C. Consequently, the sealed region of the blood vessel that has been formed by the electrifying treatment is incised.

Therefore, when the first switch 8a, for example, is pressed to form a sealed region in a blood vessel, appropriate gripping pressures are continuously applied between the electrode surface 24a of the first treatment surface 12a and the electrode surfaces 42a and 44a of the second treatment surface 14a from the initial to terminal stages of the treatment. Consequently, a sealed region is appropriatealy formed in the blood vessel.

Furthermore, when the second switch 8b is pressed to form a sealed region in a blood vessel and incise the sealed region, the sealed region is appropriately formed in the blood vessel in the same manner as when the first switch 8a is pressed. The heater 25 is energized to generate heat and the generated heat is transferred through the electrode surface 24a of the electrode 24 to the sealed region in the blood vessel, thereby incising the sealed region.

The example illustrated in FIGS. 4A and 4B has been described on the assumption that the area of contact between the electrode surface 24a of the electrode 24 of the first treatment surface 12a and the protrusion 36d of the planar portion 36 of the second treatment surface 14a is small in the widthwise directions W. The area of contact between the electrode surface 24a of the electrode 24 of the first treatment surface 12a and the protrusion 36d of the planar portion 36 of the second treatment surface 14a may be larger in the widthwise directions W. In this case, the blunter the planar portion 36 of the second treatment surface 14a, the smaller the pressure that it is able to apply to the biotissue per unit area. Therefore, the blunter the shape of the protrusion 36d, the more difficult it is for the planar portion 36 of the second treatment surface 14a to incise the biotissue.

Therefore, by appropriately setting the shape of the protrusion 36d of the planar portion 36 of the second treatment surface 14a, it is possible to adjust the coagulating performance or sealing performance and the incising performance for a biotissue. The coagulating performance or sealing performance and the incising performance for a biotissue are affected by various factors including the biotissue itself, the electric power applied to the electrodes 24 and 34, the temperature to which the heater 25 is heated, the thermal conductivity of the electrode 24, etc.

### Third Embodiment

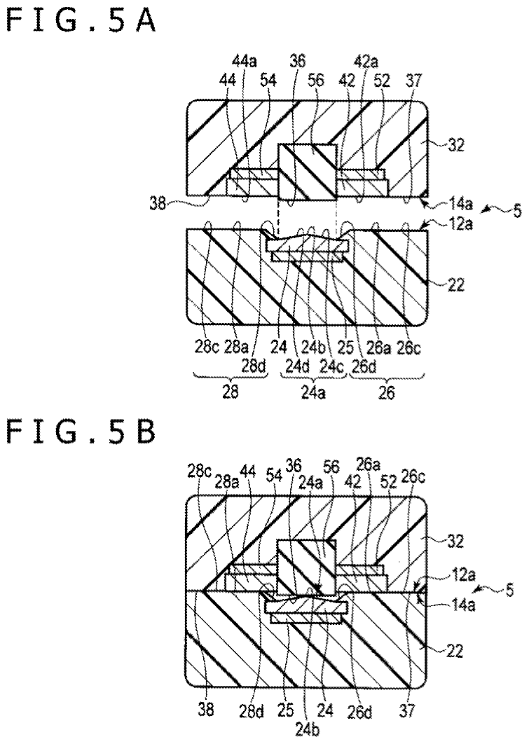

A third embodiment will be described hereinafter with reference to FIGS. 5A and 5B.

According to the first embodiment and the second embodiment, the example in which the electrode surface 24a of the first treatment surface 12a are flat surfaces has been described. According to the present embodiment, the electrode surface of the electrode 24 that has a protrusion 24b that is not a flat surface and slanted surfaces 24c and 24d will be described by way of example hereinafter. According to the present embodiment, the heater 25 is disposed in the first treatment member 12, whereas heaters 52 and 54 are disposed in the second treatment member 14.

The first treatment surface 12a has the planar portions 26 and 28 and the electrode 24 disposed between the planar portions 26 and 28.

The electrode 24 protrudes toward the planar portion 36 of the second treatment surface 14a progressively from the outer sides toward the center in the widthwise directions W. According to the present embodiment, therefore, the first treatment surface 12a is shaped as a non-flat surface. Of the electrode surface 24a of the electrode 24, a protrusive portion or crest indicated by the numeral reference 24b which protrudes most toward the second treatment surface 14a should preferably be positioned at the center in the widthwise directions W. Of the electrode 24, the region between the protrusion 24b and the planar portion 26 is shaped as a slanted surface 24c. The region between the protrusion 24b and the planar portion 28 is shaped as a slanted surface 24d. The slanted surfaces 24c and 24d make the protrusion 24b of the electrode 24 protrude toward the planar portion 36 of the second treatment surface 14a. Therefore, the electrode surface 24a is substantially V-shaped. The protrusion 24b should preferably extend continuously from nearly the distal end to nearly the proximal end of the first treatment surface 12a along the longitudinal axis L. The protrusion 24b can abut against the planar portion 36 of the second treatment surface 14a.

The planar portion 26 of the first treatment surface 12a has a slanted surface 26d lying between the abutment surface 26a and the slanted surface 24c of the electrode 24. The planar portion 28 of the first treatment surface 12a has a slanted surface 28d lying between the abutment surface 28a and the slanted surface 24d of the electrode 24. The slanted surface 26d makes the abutment surface 26a of the planar portion 26 protrude toward the second treatment surface 14a with respect to the boundary position between the slanted surface 24c of the electrode surface 24a and the slanted surface 26d. The slanted surface 28d makes the abutment surface 28a of the planar portion 28 protrude toward the second treatment surface 14a with respect to the boundary position between the slanted surface 24d of the electrode surface 24a and the slanted surface 28d. According to the present embodiment, therefore, the first treatment surface 12a is shaped as a non-flat surface.

The second treatment surface 14a has planar portions, i.e., second insulative surfaces, 36, 37, and 38 and a second electrode 34 divided into a plurality of electrode surfaces 42a and 44a. The planar portion 36 is in the form of a pad 56. The pad 56 extends along the longitudinal axis L in the second treatment surface 14a. The pad 56 is electrically insulative. The pad 56 is heat-resistant. The pad 56 should preferably be made of a softer material than the jaw 32.

The planar portion 36 of the second treatment surface 14a protrudes toward the first treatment surface 12a with respect to the electrode surface 42a that is adjacent to the planar portion 36 in the first direction W1 of the widthwise directions W and the electrode surface 44a that is adjacent to the planar portion 36 in the second direction W2 of the widthwise directions W. According to the present embodiment, therefore, the second treatment surface 14a is shaped as a non-flat surface.

The distance that the planar portion 36 protrudes with respect to the electrode surfaces 42a and 44a is substantially constant at any positions from the outer side toward the center in the widthwise directions W. The planar portion 36 can abut against the protrusion 24b of the electrode surface 24a of the first treatment surface 12a. When the protrusion 24b of the electrode 24 abuts against the planar portion 36 of the second treatment surface 14a, the abutment surface 26a of the planar portion 26 and the electrode surface 42a of the electrode member 42 abut against each other, and the abutment surface 28a of the planar portion 28 and the electrode surface 44a of the electrode member 44 abut against each other.

The abutment surface 26c of the first treatment surface 12a and the planar portion 37 of the second treatment surface 14a abut against each other in a planar fashion. The abutment surface 28c of the first treatment surface 12a and the planar portion 38 of the second treatment surface 14a abut against each other in a planar fashion.

As illustrated in FIGS. 5A and 5B, the slanted surface 26d and the electrode surface 42a, and the slanted surface 28d and the electrode surface 44a should preferably face each other along the directions in which the first treatment surface 12a and the second treatment surface 14a are opened and closed, i.e., the directions perpendicular to both the longitudinal axis L and the widthwise directions W. On the other hand, the slanted surface 24c of the electrode surface 24a and the electrode surface 42a, and the slanted surface 24d of the electrode surface 24a and the electrode surface 44a should preferably not face each other along the directions in which the first treatment surface 12a and the second treatment surface 14a are opened and closed.

A heater 52 is disposed on a reverse side of the electrode member 42 of the second electrode 34, and a heater 54 is disposed on a reverse side the electrode member 44 of the second electrode 34. The heater 52 is installed in a position shifted in the first direction W1 from the center in the widthwise directions W perpendicular to the longitudinal axis L, on the side of the electrode member 42 of the second electrode 34 opposite the electrode surface 42a. The heater 54 is installed in a position shifted in the second direction W2 from the center in the widthwise directions W perpendicular to the longitudinal axis L, on the side of the electrode member 44 of the second electrode 34 opposite the electrode surface 44a. Electric power is applied to the heaters 52 and 54 at the same time that electric power is applied to the heater 25. When the heater 52 is energized to generate heat, the heat generated by the heater 52 is transferred to the electrode surface 42a. When the heater 54 is energized to generate heat, the heat generated by the heater 54 is transferred to the electrode surface 44a.

Next, operation of the treatment tool 2 according to the present embodiment will be described hereinafter.

According to the present embodiment, in the same way as described in the first embodiment, when the first switch 8a is pressed, the high-frequency power supply 3a supplies electric power to electrodes 24 and 34, coagulating a biotissue or sealing a blood vessel. When the second switch 8b is pressed, for example, the high-frequency power supply 3a supplies electric power to the electrodes 24 and 34, and the heater power supply 3b supplies electric power to the heaters 25, 52, and 54. Therefore, according to the present embodiment, an example in which when the second switch 8b is pressed, the heater power supply 3b supplies electric power to the heaters 25, 52, and 54 to cause the heaters 25, 52, and 54 to generate heat, incising a coagulated area immediately after the coagulated area is formed, or incising a sealed area immediately after the sealed area is formed will be described hereinafter.

The heater power supply 3b supplies electric power to the heaters 25, 52, and 54 to generate heat, assisting in coagulating a biotissue or sealing a blood vessel with high-frequency output. The heater 25 is able to increase the temperature of the electrode surface 24a of the first electrode 24 with respect to the temperature thereof at the time an electric current is passed between the first electrode 24 and the second electrode 34, i.e., the electrode members 42 and 44. The heaters 52 and 54 are able to increase the temperature of the electrode surfaces 42a and 44a of the second electrode 34 with respect to the temperature thereof at the time an electric current is passed between the first electrode 24 and the second electrode 34, i.e., the electrode members 42 and 44.

When the second treatment surface 14a is brought into abutment against the first treatment surface 12a, the protrusion 24b of the electrode surface 24a and the planar portion 36 abut against each other, the abutment surface 26a and the electrode surface 42a abut against each other in a planar fashion, the abutment surface 28a and the electrode surface 44a abut against each other in a planar fashion, the abutment surface 26c and the planar portion 37 abut against each other in a planar fashion, and the abutment surface 28c and the planar portion 38 abut against each other in a planar fashion. Furthermore, when the second treatment surface 14a is brought into abutment against the first treatment surface 12a, gaps are defined between the slanted surfaces 24c and 26d and the planar portion 36 as well as the electrode surface 42a and between the slanted surfaces 24d and 28d and the planar portion 36 as well as the electrode surface 44a.

Therefore, when the first switch 8a or the second switch 8b is pressed to cause a high-frequency current to flow between the first electrode 24 and the second electrode 34, a short circuit is prevented from developing between the first electrode 24 and the second electrode 34. Of the electrode surface 42a, the area closer to the center in the widthwise directions W faces the slanted surface 26d along the directions in which the first treatment surface 12a and the second treatment surface 14a are opened and closed. Of the electrode surface 44a, the area closer to the center in the widthwise directions W faces the slanted surface 28d along the directions in which the first treatment surface 12a and the second treatment surface 14a are opened and closed. The slanted surface 24c of the electrode surface 24a and the electrode surface 42a are close to each other, and the slanted surface 24d of the electrode surface 24a and the electrode surface 44a are close to each other.

When the second treatment surface 14a is brought into abutment against the first treatment surface 12a, the protrusion 24b of the electrode surface 24a at the center in the widthwise directions W and the planar portion 36 abut against each other, the abutment surface 26a spaced from the center in the first direction W1 and the electrode surface 42a abut against each other in a planar fashion, and the abutment surface 28a spaced from the center in the second direction W2 and the electrode surface 44a abut against each other in a planar fashion. In particular, the abutment surface 26a and the electrode surface 42a, and the abutment surface 28a and the electrode surface 44a abut against each other in a planar fashion. Therefore, since the abutment surface 26a and the electrode surface 42a, and between the abutment surface 28a and the electrode surface 44a, of the first treatment surface 12a and the second treatment surface 14a of the treatment portion 5 of the treatment tool 2 according to the present embodiment, abut against each other in a planar fashion, there are no gaps therebetween along the directions in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

Furthermore, the abutment surface 26c and the planar portion 37 abut against each other in a planar fashion, and the abutment surface 28c and the planar portion 38 abut against each other in a planar fashion. Therefore, since the abutment surfaces 26a and 26c and the electrode surface 42a as well as the planar portion 37, and the abutment surfaces 28a and 28c and the electrode surface 44a as well as the planar portion 38 abut against each other in a planar fashion, there are no gaps therebetween along the directions in which the first treatment surface 12a and the second treatment surface 14a are opened and closed. Consequently, even if a tissue gripped between the first treatment surface 12a and the second treatment surface 14a is a thin tissue, the gripping pressure is transmitted to the tissue.

There are no spacers present between the abutment surfaces 26a and 26c and the electrode surface 42a and the planar portion 37, and between the abutment surfaces 28a and 28c and the electrode surface 44a and the planar portion 38. Therefore, the gripping pressure under which a biotissue as a treatment target is gripped along the widthwise directions W is restrained from varying greatly between the abutment surfaces 26a and 26c and the electrode surface 42a as well as the planar portion 37, and between the abutment surfaces 28a and 28c and the electrode surface 44a as well as the planar portion 38. Moreover, a biotissue as a treatment target can easily be gripped in a larger area between the abutment surfaces 26a and 26c and the electrode surface 42a as well as the planar portion 37, and between the abutment surfaces 28a and 28c and the electrode surface 44a as well as the planar portion 38.

While the first treatment surface 12a and the second treatment surface 14a of the treatment portion 5 of the treatment tool 2 according to the present embodiment are held in abutment against each other, therefore, there are regions in which no gaps are present along the directions in which the first treatment surface 12a and the second treatment surface 14a are opened and closed and which are perpendicular to the longitudinal axis L and the widthwise directions W. Consequently, even if a tissue gripped between the first treatment surface 12a and the second treatment surface 14a is a thin tissue, the gripping pressure is reliably transmitted to the tissue.

A treatment, i.e., an electrifying treatment, for passing a high-frequency current through a blood vessel, not illustrated, to form a sealed region therein, using the treatment portion 5 of the treatment tool 2 according to the present embodiment will be described by way of example hereinafter.

In the same manner as described in the first embodiment, a blood vessel as a treatment target is gripped between the first treatment surface 12a and the second treatment surface 14a. The blood vessel is gripped while in contact with both the first treatment surface 12a and the second treatment surface 14a.

There are gaps defined between the slanted surfaces 24c and 26d and the planar portion 36 as well as the electrode surface 42a, and between the slanted surfaces 24d and 28d and the planar portion 36 as well as the electrode surface 44a. A blood vessel is gripped between the protrusion 24b of the electrode surface 24a and the planar portion 36, between the abutment surface 26a and the electrode surface 42a, and between the abutment surface 28a and the electrode surface 44a. Therefore, the blood vessel is held in contact with both the electrode 24 of the first treatment surface 12a and the electrode 34 of the second treatment surface 14a while under the gripping pressure.

When the user presses the first switch 8a, the blood vessel between the first electrode 24 and the second electrode 34 is progressively dehydrated and dried, and becomes thinner. Therefore, the distance between the first treatment surface 12a and the second treatment surface 14a becomes smaller as the blood vessel is thinner.

Consequently, the treatment portion 5 of the treatment tool 2 according to the present embodiment applies a maximum gripping pressure when it is about to finish the treatment to seal the blood vessel. Consequently, an appropriate sealed region is formed in the blood vessel.

The example in which the treatment is performed by pressing the first switch 8a to supply electric power from the high-frequency power supply 3a to the electrodes 24 and 34 to form a sealed region in a blood vessel has been described hereinbefore. A similar treatment is carried out to coagulate a treatment target of a biotissue.

Next, an example in which a sealed region is formed in a blood vessel and the formed sealed region is incised using the treatment tool 2 according to the present embodiment will be described hereinafter.

For incising the blood vessel, it is preferable to energize the heaters 25, 52, and 54 to generate heat and apply the heat at a temperature in excess of 100° C., e.g., approximately 200° C., through the electrode surfaces 24a, 42a, and 44a to the blood vessel while an appropriate gripping pressure is being applied thereto.

With the treatment portion 5 of the treatment tool 2 according to the present embodiment, the protrusion 24b applies a gripping pressure to press the sealed region of the blood vessel against the planar portion 36. Even when the blood vessel is progressively thinner at the center in the widthwise directions W, the treatment portion 5 continues to apply an appropriate gripping pressure between the protrusion 24b and the planar portion 36. In this state, the heat generated by the heaters 25, 52, and 54 is transferred to the electrode surfaces 24a, 42a, and 44a. Therefore, while the appropriate pressure is being applied to the sealed region of the blood vessel, the temperature of the sealed region is increased to a temperature in excess of 100° C. Consequently, the sealed region of the blood vessel that has been formed by the electrifying treatment is incised.

Therefore, when the first switch 8a, for example, is pressed to form a sealed region in a blood vessel, appropriate gripping pressures are continuously applied between the electrode surface 24a of the first treatment surface 12a and the electrode surfaces 42a and 44a of the second treatment surface 14a from the initial to terminal stages of the treatment. Consequently, a sealed region is appropriately formed in the blood vessel.

Furthermore, when the second switch 8b is pressed to form a sealed region in a blood vessel and incise the sealed region, the sealed region is appropriately formed in the blood vessel in the same manner as when the first switch 8a is pressed. The heaters 25, 52, and 54 are energized to generate heat and the generated heat is transferred through the electrode surface 24a of the electrode 24 and the electrode surfaces 42a and 44a of the electrode 34 to the sealed region in the blood vessel, thereby incising the sealed region.

Therefore, as with the treatment tool 2 described in the first embodiment, the treatment tools 2 according to the second and third embodiments are capable of continuously applying appropriate gripping pressures to the treatment target between the treatment surfaces from the initial to terminal stages of the treatment.

In the first and second embodiments, the examples in which the single heater, i.e., heat generating body, 25 is disposed in the first treatment member 12 are described. In the third embodiment, the example in which the two heaters, i.e., heat generating bodies, 52 and 54 are disposed in the second treatment member 14 is described. Although not illustrated, no heater may be disposed in the first treatment member 12 insofar as there is a heater capable of transferring heat to the electrode surfaces 42a and 44a of the second treatment member 14.

The certain embodiments have hereinbefore been described in specific detail with reference to the drawings. The disclosed technology is not limited to the embodiments described hereinbefore, but covers all embodiments that may be carried out without departing from the scope of the invention.

In sum, the disclosed technology is directed to an elongated treatment tool having a treatment portion disposed on a longitudinal axis thereof. The treatment portion includes a first treatment surface having a first electrically insulative surface and a first electrically conductive electrode extending along the longitudinal axis at a center of width of the first insulative surface. A second treatment surface having a second electrically insulative surface and a second electrically conductive electrode extending along the longitudinal axis of the second insulative surface. The second treatment surface is rotatable relatively with respect to the first treatment surface about a turn shaft perpendicular to the longitudinal axis and parallel to the widthwise directions perpendicular to the longitudinal axis. A heater is disposed on the first electrode for generating heat when supplied with electric power. When the second treatment surface is brought into abutment against the first treatment surface, the second electrically conductive electrode and the first electrically insulative surface abut against one another thereby to keep the first electrically conductive electrode and the second electrically conductive electrode spaced from one another.

The heater is located centrally in the widthwise directions perpendicular to the longitudinal axis. The respective first and second treatment surfaces each of which extends along the longitudinal axis. The first treatment surface and the second treatment surface have respective abutment surfaces that are electrically insulative and are disposed when the second treatment surface is brought into abutment against the first treatment surface. The first treatment surface and the second treatment surface are disposed in areas outside of the center in the widthwise directions perpendicular to the longitudinal axis. The first electrode includes a first electrode surface. The second electrode includes a second electrode surface. The first insulative surface of the first treatment surface protrudes toward the second treatment surface beyond the first electrode surface. The second insulative surface of the second treatment surface protrudes toward the first treatment surface beyond the second electrode surface. The first electrode includes a first electrode surface. The second electrode includes a second electrode surface. The first treatment surface extends along the longitudinal axis. The heater is disposed on a side of the first electrode that is opposite to the first electrode surface. The first insulative surface protrudes toward the second treatment surface beyond the first electrode surface or the second insulative surface protrudes toward the first treatment surface beyond the second electrode surface.

The heater is disposed in vicinity of the center in the widthwise directions perpendicular to the longitudinal axis on the side of the first electrode that is opposite to the first electrode surface. The first electrode includes a first electrode surface. The second electrode includes a second electrode surface. The first insulative surface includes a flat surface. The second electrode surface includes a flat surface. The first insulative surface and the second electrode surface of the second treatment surface are capable of abutting against one another in a planar orientation. The first treatment surface extends along the longitudinal axis. The first treatment surface includes a pair of electrically insulative first planar portions extending toward outer edges in a first direction and from the center along the widthwise directions perpendicular to the longitudinal axis in a second direction. The second treatment surface includes a pair of electrically insulative second planar portions extending toward the outer edges in the first direction and from the center along the widthwise directions in the second direction. When the second treatment surface is brought into abutment against the first treatment surface, the pair of electrically insulative first planar portions and the pair of electrically insulative second planar portions are capable of abutting respectively against each other in a planar orientation. The pair of electrically insulative first planar portions and the pair of electrically insulative second planar portions are slanted with respect to the first direction and the second direction, respectively.

Another aspect of the disclosed technology is directed to a treatment system having an energy source apparatus and an elongated treatment tool. The elongated treatment tool is configured to be attached to the energy source apparatus to receive electrical energy. The elongated treatment tool includes a treatment portion disposed on a longitudinal axis thereof and used to grip a treatment target such as a biological tissue. The treatment portion includes a first treatment surface having a first electrically insulative surface and a first electrically conductive electrode each of which extends along the longitudinal axis of the first electrically insulative surface. A second treatment surface having a second electrically insulative surface and a second electrically conductive electrode each of which extends along the longitudinal axis of the second insulative surface. The second treatment surface is rotatable with respect to the first treatment surface about a turn shaft perpendicular to the longitudinal axis. A heater is disposed on the first electrode to generate heat when supplied with electric power. When the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

A further aspect of the disclosed technology is directed to a treatment system includes an energy source apparatus having respective high frequency and heater power supplies and an elongated treatment tool configured to be attached to the energy source apparatus to receive electrical energy. The elongated treatment tool includes a main body, a shaft, and a treatment portion all of which are attached to one another and are disposed on a longitudinal axis thereof. The treatment portion is used to grip a treatment target so as to apply appropriate gripping pressure to a point where the treatment target is to coagulate and to form a sealed region therein from an initial stage to a terminal stage of the treatment. The treatment portion includes a first treatment surface having a first electrically insulative surface and a first electrically conductive electrode each of which extends along the longitudinal axis of the first electrically insulative surface. A second treatment surface having a second electrically insulative surface and a second electrically conductive electrode each of which extends along the longitudinal axis of the second insulative surface. The second treatment surface is rotatable with respect to the first treatment surface about a turn shaft perpendicular to the longitudinal axis. The heater is disposed on the first electrode to generate heat when supplied with the heater power supply. When the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An elongated treatment tool having a treatment portion disposed on a longitudinal axis thereof,
the treatment portion including:
a first treatment surface having a first electrically insulative surface and a first electrically conductive electrode extending along the longitudinal axis at a center of width of the first insulative surface;
a second treatment surface having a second electrically insulative surface and a second electrically conductive electrode extending along the longitudinal axis of the second insulative surface, the second treatment surface being rotatable relatively with respect to the first treatment surface about a turn shaft perpendicular to the longitudinal axis and parallel to the widthwise directions perpendicular to the longitudinal axis; and
a heater disposed on the first electrode , for generating heat when supplied with electric power, wherein
when the second treatment surface is brought into abutment against the first treatment surface, the second electrically conductive electrode and the first electrically insulative surface abut against one another thereby to keep the first electrically conductive electrode and the second electrically conductive electrode spaced from one another.

2. The elongated treatment tool of claim 1, wherein the heater located centrally in the widthwise directions perpendicular to the longitudinal axis.

3. The elongated treatment tool of claim 1, wherein
the first treatment surface and the second treatment surface each of which extends along the longitudinal axis; and
the first treatment surface and the second treatment surface have respective abutment surfaces that are electrically insulative and being disposed when the second treatment surface is brought into abutment against the first treatment surface.

4. The elongated treatment tool of claim 1, wherein the first treatment surface and the second treatment surface are disposed in areas outside of the center in the widthwise directions perpendicular to the longitudinal axis

5. The elongated treatment tool of claim 1, wherein
the first electrode includes a first electrode surface;
the second electrode includes a second electrode surface;
the first insulative surface of the first treatment surface protrudes toward the second treatment surface beyond the first electrode surface; and
the second insulative surface of the second treatment surface protrudes toward the first treatment surface beyond the second electrode surface.

6. The elongated treatment tool of claim 1, wherein
the first electrode includes a first electrode surface;
the second electrode includes a second electrode surface;
the first treatment surface extends along the longitudinal axis;
the heater being disposed on a side of the first electrode that is opposite to the first electrode surface; and
the first insulative surface protrudes toward the second treatment surface beyond the first electrode surface or the second insulative surface protrudes toward the first treatment surface beyond the second electrode surface.

7. The elongated treatment tool of claim 1, wherein the heater is disposed in vicinity of the center in the widthwise directions perpendicular to the longitudinal axis on the side of the first electrode that is opposite to the first electrode surface.

8. The elongated treatment tool of claim 1, wherein
the first electrode includes a first electrode surface;
the second electrode includes a second electrode surface;
the first insulative surface includes a flat surface;
the second electrode surface includes a flat surface; and
the first insulative surface and the second electrode surface of the second treatment surface are capable of abutting against one another in a planar orientation.

9. The elongated treatment tool of claim 1, wherein
the first treatment surface extends along the longitudinal axis;
the first treatment surface includes a pair of electrically insulative first planar portions extending toward outer edges in a first direction and from the center along the widthwise directions perpendicular to the longitudinal axis in a second direction;
the second treatment surface includes a pair of electrically insulative second planar portions extending toward the outer edges in the first direction and from the center along the widthwise directions in the second direction; and
when the second treatment surface is brought into abutment against the first treatment surface, the pair of electrically insulative first planar portions and the pair of electrically insulative second planar portions are capable of abutting respectively against each other in a planar orientation.

10. The elongated treatment tool of claim 9, wherein
the pair of electrically insulative first planar portions and the pair of electrically insulative second planar portions are slanted with respect to the first direction and the second direction, respectively.

11. A treatment system comprising:
an energy source apparatus; and
an elongated treatment tool configured to be attached to the energy source apparatus to receive electrical energy, the elongated treatment tool includes a treatment portion being disposed on a longitudinal axis thereof and being used to grip a treatment target and wherein the treatment portion includes:
a first treatment surface having a first electrically insulative surface and a first electrically conductive electrode each of which extends along the longitudinal axis of the first electrically insulative surface,
a second treatment surface having a second electrically insulative surface and a second electrically conductive electrode each of which extends along the longitudinal axis of the second insulative surface, the second treatment surface being rotatable with respect to the first treatment surface about a turn shaft perpendicular to the longitudinal axis, and
a heater disposed on the first electrode to generate heat when supplied with electric power wherein
when the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

12. The treatment system of claim 11, wherein the respective first and second electrically conductive electrodes include respective first and second electrically conductive electrode surfaces wherein
the first electrically insulative surface protrudes toward the second treatment surface beyond the first electrically conductive electrode surface and
the second electrically insulative surface protrudes toward the first treatment surface beyond the second electrically conductive electrode surface.

13. The treatment system of claim 11, wherein the respective first and second electrically conductive electrodes include respective first and second electrically conductive electrode surfaces wherein the heater is disposed on a side of the first electrically conductive electrode.

14. A treatment system comprising:
an energy source apparatus having respective high frequency and heater power supplies; and
an elongated treatment tool configured to be attached to the energy source apparatus to receive electrical energy, the elongated treatment tool includes a main body, a shaft, and a treatment portion all of which being attached to one another and being disposed on a longitudinal axis thereof, the treatment portion being used to grip a treatment target so as to apply appropriate gripping pressure to a point where the treatment target is to coagulate and to form a sealed region therein from an initial stage to a terminal stage of the treatment, the treatment portion includes:
a first treatment surface having a first electrically insulative surface and a first electrically conductive electrode each of which extends along the longitudinal axis of the first electrically insulative surface,
a second treatment surface having a second electrically insulative surface and a second electrically conductive electrode each of which extends along the longitudinal axis of the second insulative surface, the second treatment surface being rotatable with respect to the first treatment surface about a turn shaft perpendicular to the longitudinal axis, and
a heater disposed on the first electrode to generate heat when supplied with the heater power supply wherein
when the second treatment surface is brought into abutment against the first treatment surface, the second electrically conductive electrode and the first electrically insulative surface abut against one another thereby to keep the first electrically conductive electrode and the second electrically conductive electrode being spaced apart from one another.

15. The treatment system of claim 14, wherein the treatment target is a biological tissue.
